(19) United States
(12) Patent Application Publication
(10) Pub. No.: US 2009/0221857 A1
(43) Pub. Date: Sep. 3, 2009

(54) PROCESS FOR THE PREPARATION OF TAMSULOSIN AND RELATED COMPOUNDS

(76) Inventor: Ignasi Auquer Pedemonte, Girona (ES)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 12/091,237

(22) PCT Filed: Oct. 27, 2006
(86) PCT No.: PCT/IB06/04231

§ 371 Date: Oct. 30, 2008

Related U.S. Application Data
(60) Provisional application No. 60/730,865, filed on Oct. 28, 2005.

(51) Int. Cl. C07C 321/28

(52) U.S. Cl. 564/341

(57) ABSTRACT

The invention relates, in general, to the preparation of (R)(−)tamsulosin free base by reaction of (R)-5-(2-aminopropyl)-2-methoxybenzenesulfonamide or an addition salt thereof with 1-(2-bromoethoxy)-2-ethoxybenzene in a polar aprotic solvent in the presence of an organic base. More particularly, the invention relates to a process for preparing pure solid crystalline (R)(−)tamsulosin in its free base form as a precursor for the production of (R)(−)tamsulosin hydrochloride.

### CROSS REFERENCE TO RELATED APPLICATIONS

This application claims priority to U.S. Provisional Application No. 60/730,865, filed Oct. 28, 2005, which is expressly incorporated herein by reference in its entirety.

### BACKGROUND OF THE INVENTION

1. Field of the Invention

The invention relates, in general, to the preparation of tamsulosin free base. More particularly, the invention relates to a process for preparing pure solid crystalline tamsulosin in its free base form as a precursor for the production of tamsulosin hydrochloride.

2. Relevant Background

Tamsulosin hydrochloride is a commercially marketed pharmaceutically active substance known to be useful for the treatment of prostatic disorders, such as benign prostatic hyperplasia (BPH), as it is believed to operate as an antagonist of alpha1 A adrenoceptors in the prostate. Tamsulosin hydrochloride, having an empirical formula of C₂₀H₂₈N₂O₅S•HCl and a molecular weight (“MW”) of 444.98, can be readily obtained from tamsulosin. Tamsulosin (i.e., tamsulosin free base) is the international commonly accepted name for 5-[(2R)-2-[[2-(2-ehtoxyphenoxy)ethyl]amino]propyl]-2-methoxybenzene-sulfonamide, which is represented in formula (I).

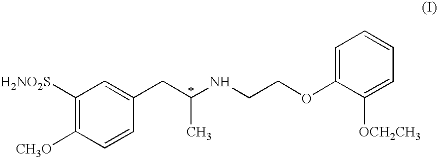

Notably, as depicted in formula (I), tamsulosin appears in two enantiomeric forms (R and S), with the R form being the more commercially desirable enantiomer due to its pharmacological activity.

There are various known mechanisms for producing tamsulosin. For example, U.S. Pat. No. 4,731,478 (the “'478 patent”) (equivalent to EP 34432) describes several processes by which tamsulosin can be produced. A first process of the '478 patent includes the conversion of a hydroxylated analogue into the desired sulfonamide via a chloro-analogue. The hydroxy analogue of tamsulosin is a compound having a structure as depicted in formula (II).

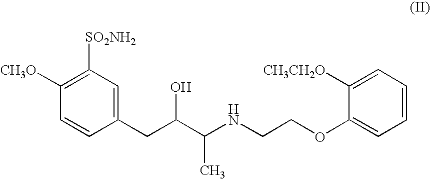

A second process includes a reductive amination of a benzylmethylketone compound with an appropriately substituted phenoxyethylamine. According to this second process, the benzylmethylketone can be represented by formula (III), and the phenoxyethylamine can be represented by formula (IV).

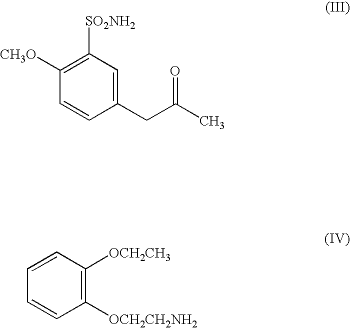

Unfortunately, the two processes described above produce racemic (R, S) tamsulosin, requiring the additional step of isolating the R enantiomer form if the product is to be used for pharmaceutical purposes. While optical purification of racemic tamsulosin is possible, it is generally undesirable commercially.

The '478 patent, therefore, describes a process capable of producing optically pure enantiomer forms of tamsulosin. This process includes obtaining optically pure 5-((2-amino-2-methyl)ethyl)-2-methoxybenzenesulfonamide, as depicted in formula (V), and reacting it with 2-(o-ethoxyphenoxy)ethyl bromide, as depicted in formula (VI), to form the corresponding (R) or (S) tamsulosin enantiomer. See Examples 33(a) and 33(b) of the '478 patent.

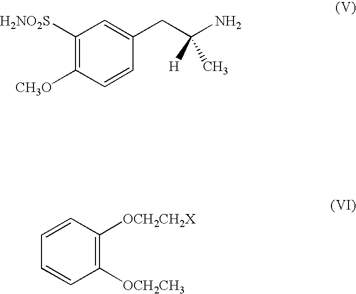

Thus (R)-5-((2-amino-2-methyl)ethyl)-2-methoxybenzenesulfonamide could be used to produce (R)-tamsulosin or (S)-tamsulosin. In particular, this process described in the '478 patent for preparing optically pure tamsulosin involves mixing the starting compounds in ethanol and reacting them at reflux temperature for about 16 hours to produce a crude oily tamsulosin product. The crude tamsulosin product includes unused reactants and undesirable by-products of the reaction. This crude product thereafter requires a purification procedure by means of silica-gel column chromatography to isolate the tamsulosin before it can be converted into the desired corresponding hydrochloride.

European patent application EP 380 144A (the “'144A application”) describes a similar process for producing optically pure tamsulosin that involves reacting the compounds of formula (V) and formula (VI) at either room temperature, elevated temperature, or under reflux and either in the absence of solvent or in an organic solvent, such as benzene, toluene, xylene, dimethylformamide, dichloromethane, methanol or ethanol. The '144A application also describes that, optionally, a secondary or tertiary amine (e.g., pyridine, picoline, N,N-dimethylaniline, N-methylmorpholine, trimethylamine, triethylamine or dimethylamine) or inorganic bases (e.g., potassium carbonate, sodium carbonate or sodium bicarbonate) can additionally be used to ensure a smooth reaction. As with the process described in the '478 patent, this process requires purification of crude tamsulosin product thereafter, before further converting it into tamsulosin hydrochloride.

WO 03/037851 (Ex. 2(A)) discloses that the above reaction can be carried out in dimethylformamide as a solvent at 80-85° C. for 4 hours after which the solvent is removed by vacuum distillation. Water is then added to the solid residue, and the mixture is heated to 80-90° C. with stirring for 2 hours. The mixture is then cooled to room temperature. The resulting crystals are next isolated by filtration, suspended in water and heated to 80-90° C. with stirring for 2 hours. The crystals are then filtered, washed with water and dried to yield a racemic mixture of tamsulosin base.

ES 2000382 (Ex. 9) discloses that the above reaction can be carried out in dimethylformamide as a solvent at 60° C. for 6 hours after which the solvent is removed by vacuum distillation. Water is then added to the solid residue and the mixture is heated to reflux with stirring for 30 minutes. The mixture is then cooled to 5° C. and stirred for 1 hour. The resulting crystals are then isolated by filtration and re-crystallized in isopropanol to yield tamsulosin base.

These known processes for producing tamsulosin, and, in particular, (R)-tamsulosin, are not optimal for industrial implementation because they lead to the presence of high amounts of undesired byproducts which in turn makes it necessary to use economically disadvantageous purification processes to isolate the product to the extent required by quality specifications, such as to pharmaceutical grade product. Thus, there remains a need for improved processes for producing tamsulosin.

### SUMMARY OF THE INVENTION

The invention relates, in general, to the preparation of tamsulosin free base. More particularly, the invention relates to a process for preparing pure solid crystalline tamsulosin in its free base form as a precursor for the production of tamsulosin hydrochloride.

The process for preparing tamsulosin includes reacting a compound of formula (V):

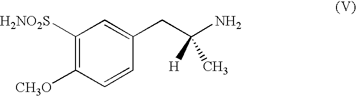

as the free base or as an addition salt thereof with an organic or inorganic acid, with a compound of formula (VI), wherein X is a halogen atom:

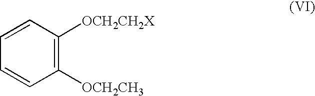

in the presence of a neutralizing agent, and isolating crystalline (R)-tamsulosin free base and prior to converting the same into tamsulosin HCl.

The tamsulosin free base obtained can be transformed by conventional methodologies, if desired, into a pharmaceutically acceptable acid addition salt, solvate, hydrate or clathrate thereof.

All of the compounds to be used in the processes of the invention are commercially available. Alternatively, optically pure (R )-5-((2-amino-2-methyl)ethyl)-2-methoxybenzenesulfonamide (V) can be obtained by treating racemic 5-((2-amino-2-methyl)ethyl)-2-methoxybenzenesulfonamide (not shown) with a sulfonic chiral acid such as (1R)-(−)-10-camphorsulphonic acid in an alkanol solvent.

The precipitated salt, which is substantially enriched by the desired enantiomer, can be recrystallized from a mixture of an alkanol solvent and water to liberate the enriched enantiomer to form the salt. The liberation step includes treatment of the salt (in solid, suspended or dissolved state) with an organic or inorganic base. The base should be stronger than the basicity of desired enantiomer. Generally, the liberation of the desired enantiomer from the enriched salt proceeds by contacting the salt with an equivalent of a suitable base, e.g., metal hydroxides or ammonia, in a proper solvent, advantageously in water. The so formed free base of the desired enantiomer is normally then isolated by ordinary methods. If water has been employed as solvent for neutralization, the desired enantiomer base precipitates as a solid and can be isolated by filtration or centrifugation.

Preferably, the halogen atom, X, of compound VI is bromine. Also, it is preferable that compound V is used as its addition salt with an acid selected from hydrochloric acid, hydrofluoric acid, hydrobromic acid, hydroiodic acid, methanesulfonic acid, trifluoromethanesulfonic acid and/or trifluoroacetic acid, and where the acid is more preferably hydrochloric acid.

Preferably, the relative amount of compounds V and VI is close to equimolar, and more preferably with a slight excess of compound V.

A neutralizing agent is used to neutralize the hydrohalic acid (H—X) formed during the coupling reaction. The neutralizing agent can be an organic or inorganic base, preferably selected from alkali or alkaline earth metal carbonates (e.g., sodium carbonate or potassium carbonate); bicarbonates (e.g., sodium bicarbonate) or tertiary amines (e.g., triethylamine or diisopropylethylamine). In a preferred embodiment, the neutralizing agent is diisopropylethylamine.

Preferably the neutralizing agent is used in excess. More preferably, the process employs from one to two molar equivalents of a neutralizing agent based on the quantity of the starting material.

Additional embodiments of the invention include utilizing tamsulosin base made according to the described process to further produce pharmaceutically acceptable or desirable addition salts, hydrates, solvates, or clathrates of tamsulosin. Preferably, these additional embodiments include forming an acid addition salt of tamsulosin obtained from the above reaction by treating a solution or suspension of that tamsulosin with an appropriate acid. Such processes isolate the tamsulosin product by (i) extracting the tamsulosin product in an organic solvent and (ii) at least partially evaporating the organic solvent to obtain the tamsulosin product, and then reacting the isolated tamsulosin product to produce the tamsulosin salt, hydrate, solvate or clathrate.

Optionally, additional purification steps can be included without altering the invention, such as heating in an organic solvent, filtering, drying, and re-crystallizing in a hot alkanol, such as ethanol.

The invention further includes formulating tamsulosin, its salts and/or in vivo cleavable prodrugs thereof (collectively “the compounds of the invention”) prepared from tamsulosin base made according to the described process, into readily usable dosage units for the therapeutic treatment (including prophylactic treatment) of mammals, including humans.

The process for preparing tamsulosin free base described herein is advantageous for a number of reasons. In particular, the (R)-tamsulosin free base is commonly isolated as a pure white crystalline solid that is further purified by conventional methods, such as crystallization. Generally, the reaction is performed using dimethylformamide and diisopropylethylamine as a base at approximately 100° C. for approximately 90 to approximately 120 minutes. After cooling the reaction mixture to room temperature (˜25° C.), ethyl acetate and water are added to the reaction mixture. After decanting the two phases, the water phase is further extracted using ethyl acetate, and the combined organic extracts are washed once with water. Upon distilling part of the ethyl acetate, (R)-tamsulosin free base precipitates. Upon further distillation, the (R)-tamsulosin free base can be readily isolated, for example by filtration, and further purified. At this stage, the purity according to high performance liquid chromatography (HPLC) is approximately 97.62% (area).

Alternatively, the reaction can be performed using acetonitrile instead of dimethylformamide which results in the precipitation of solid (R)-tamsulosin free base crystallize from the reaction mixture. The solid (R)-tamsulosin free base can be purified by treatment with an alcohol, preferably methanol. Under such conditions, HPLC purities higher than 99.8% are achieved. It is believed that the foregoing is the first example of a process for directly isolating crystalline (R)-tamsulosin base from the reaction mixture directly.

In view of the foregoing, the processes of the invention are advantageous, for among other reasons, because:

1. Such processes enable highly pure tamsulosin free base to be prepared without the need to purify the same by column chromatography;

2. Such processes eliminate the need to remove dimethylformamide, when used as the reaction solvent, by distillation;

3. Such processes have improved yields; and

4 Such processes employ an approximately stoichiometric amount of compound V to compound VI. Previous processes employ an excess (e.g., 2 to 4 times excess) of compound V (or the corresponding racemic mixture thereof) relative to compound VI. Thus, the quantity of compound V employed to produce tamsulosin base is much lower than the quantity used by previously known processes. By reducing the amount of compound V that is required to be used, there is a concomitant cost advantage (i.e., the costs of using excess compound V) over known processes.

The various embodiments of the invention having thus been generally described, several examples will hereafter be discussed to illustrate the inventive aspects more fully.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates the X-ray powder diffractogram of (R)-tamsulosin free base prepared according to a process of the invention;

FIG. 2 illustrates the infrared spectrum (IR) of (R)-tamsulosin free base prepared according to a process of the invention;

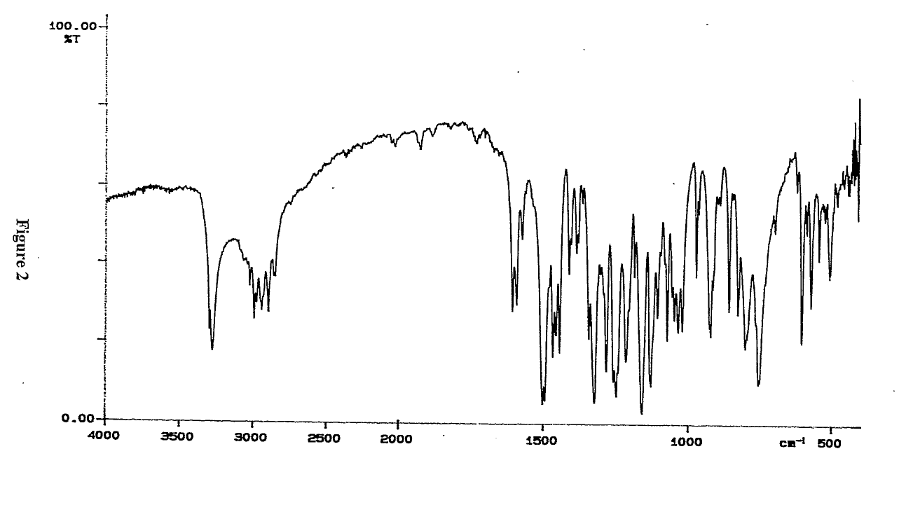

FIG. 3 illustrates the X-ray powder diffractogram of tamsulosin hydrochloride obtained from (R)-tamsulosin free base prepared according to a process of the invention;

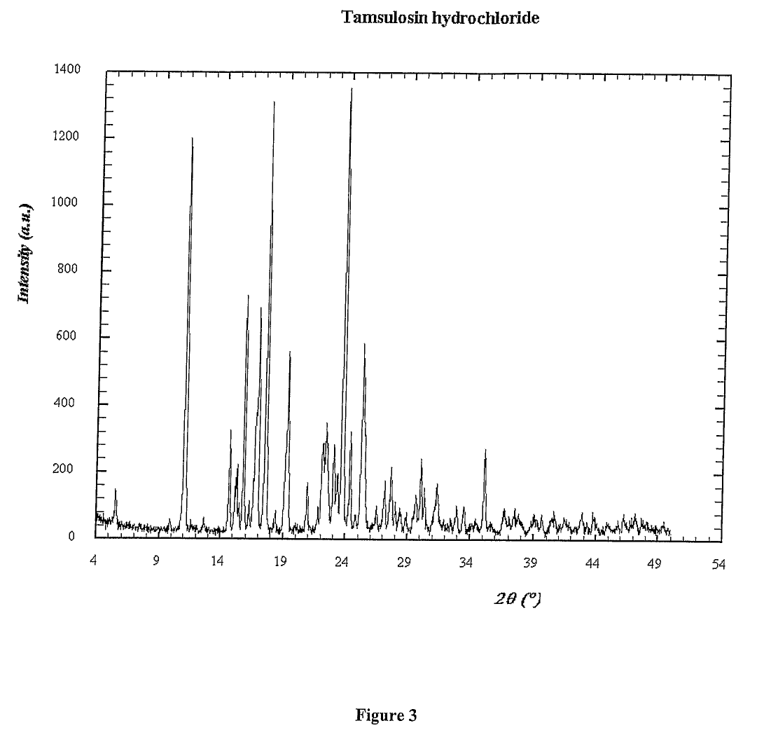

FIG. 4 illustrates the IR spectrum of tamsulosin hydrochloride obtained from (R)-tamsulosin free base prepared according to a process of the invention; and

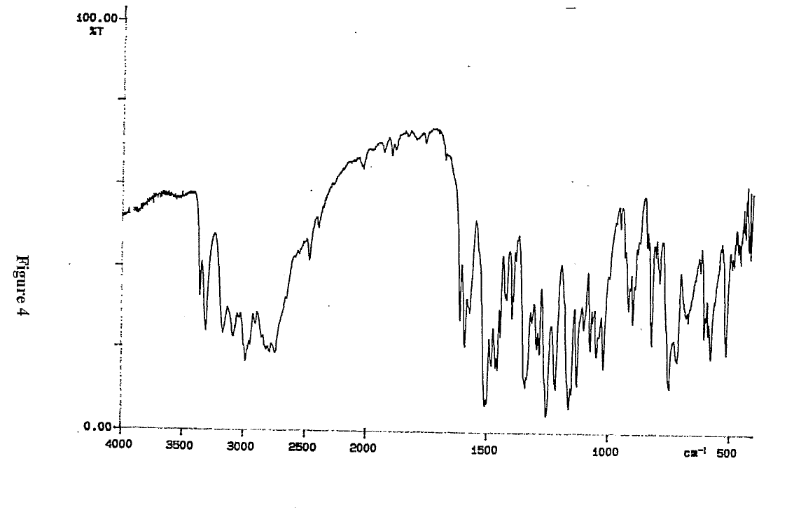

FIG. 5 illustrates the Differential Scanning Calorimetry (DSC) thermogram of tamsulosin hydrochloride obtained from (R)-tamsulosin free base prepared according to a process of the invention.

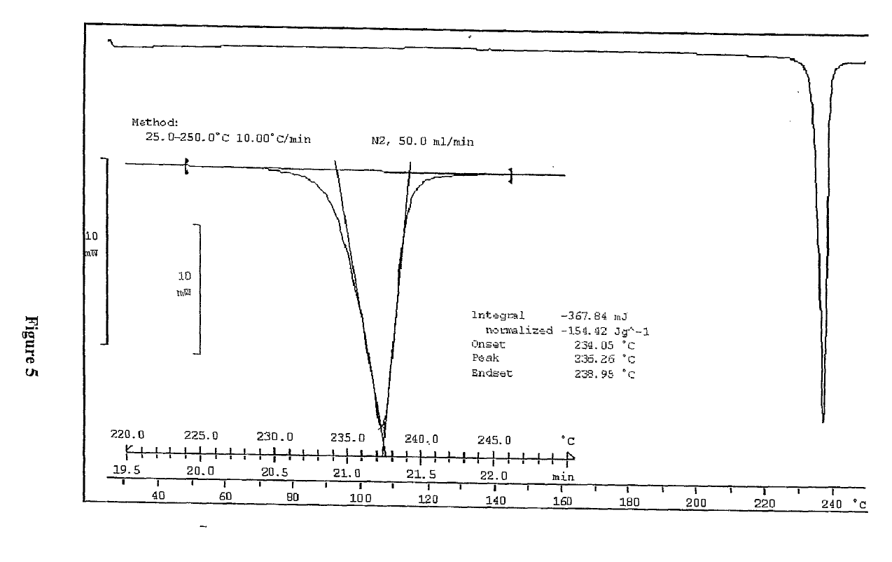

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition and as will be appreciated by one of skill in the art, the invention may be embodied as a method, system or process.

The invention relates, in general, to the preparation of tamsulosin free base. More particularly, the invention relates to a process for preparing pure solid crystalline tamsulosin in its free base form as a precursor for the production of tamsulosin hydrochloride.

The invention includes a process for preparing of (R)(−)tamsulosin free base that includes reacting (R)-5-(2-aminopropyl)-2-methoxybenzenesulfonamide or an addition salt thereof with 1-(2-bromoethoxy)-2-ethoxybenzene in a polar aprotic solvent in the presence of an organic base and in which the molar ratio of sulphonamide to the bromoethoxy compound is between approximately 0.90 and approximately 1.10.

The invention further includes a process for preparing (R)(−)tamsulosin free base that includes reacting (R)-5-(2-aminopropyl)-2-methoxybenzenesulfonamide or an addition salt thereof with 1-(2-bromoethoxy)-2-ethoxybenzene in a polar aprotic solvent in the presence of an organic base and in which the molar ratio of sulphonamide to the bromoethoxy compound is approximately 1.05.

The invention further includes a process for preparing (R)(−)tamsulosin free base in which N,N-dimethylformamide is used as a reaction solvent, and further includes a process in which the N,N-dimethylformamide is not removed by distallation.

The invention further includes a process for preparing (R)(−)tamsulosin free base in which (R)(−)tamsulosin is extracted in an organic solvent, and then partially evaporating the organic solvent to obtain crystalline (R)(−)tamsulosin free base.

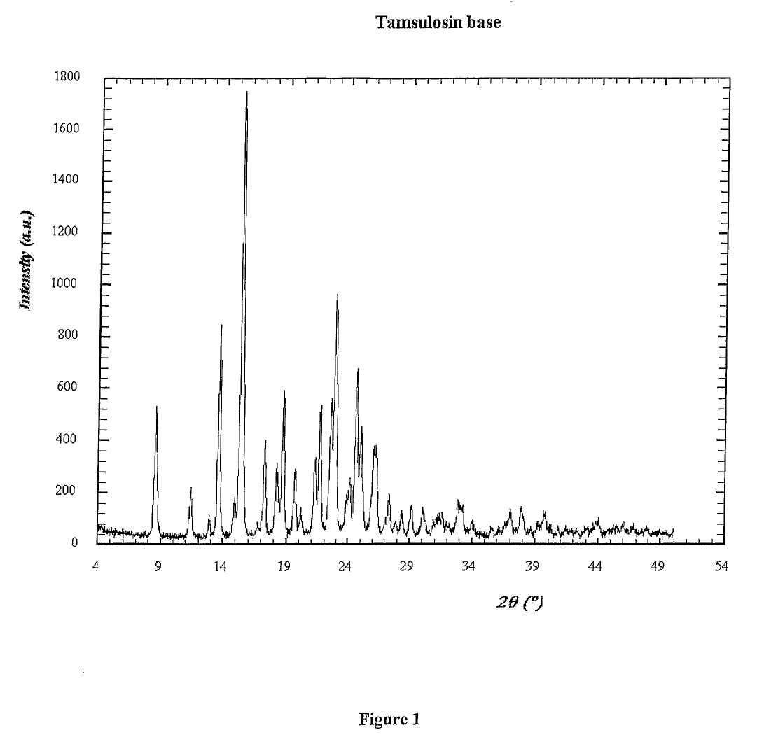

The invention further includes solid crystalline (R)(−)tamsulosin free base having an X-ray diffraction pattern (2θ) substantially similar to that of FIG. 1.

The invention further includes solid crystalline (R)-(−)tamsulosin free base having an X-ray diffraction pattern (2θ) having characteristics peaks at 8.56, 13.61, 15.38, 17.25, 18.68 and 22.85 degrees.

The invention further includes the use of solid crystalline (R)(−)tamsulosin free base described herein in a process for manufacturing (R)(−)tamsulosin hydrochloride.

The invention further includes (R)(−)tamsulosin free base having a purity higher than 97% when analyzed by reverse phase HPLC.

The invention further includes the use of (R)(−)tamsulosin free base of high purity in a process for the manufacture of (R)(−)tamsulosin hydrochloride.

The invention further includes (R)(−)tamsulosin hydrochloride, obtained from (R)-tamsulosin free base prepared according to a process of the invention, having a purity higher than 99% when analyzed by reverse phase HPLC.

The invention further includes (R)(−)tamsulosin hydrochloride, obtained from (R)-tamsulosin free base prepared according to a process of the invention, having a purity higher than 99.5% when analyzed by reverse phase HPLC.

The invention further includes formulations containing (R)(−)tamsulosin hydrochloride, obtained from (R)-tamsulosin free base prepared according to a process of the invention, having a purity higher than 99% when analyzed by reverse phase HPLC.

The invention further includes formulations containing (R)(−)tamsulosin hydrochloride, obtained from (R)-tamsulosin free base prepared according to a process of the invention, having a purity higher than 99.5% when analyzed by reverse phase HPLC.

The invention further includes (R)(−)tamsulosin hydrochloride, obtained from (R)-tamsulosin free base prepared according to a process of the invention, having a content of (S)(+)tamsulosin hydrochloride of less than 1% when analyzed by chiral HPLC.

The invention further includes (R)(−)tamsulosin hydrochloride, obtained from (R)-tamsulosin free base prepared according to a process of the invention, having a content of (S)(+)tamsulosin hydrochloride of less than 0.5% when analyzed by chiral HPLC.

The invention further includes (R)(−)tamsulosin hydrochloride, obtained from (R)-tamsulosin free base prepared according to a process of the invention, having a content of (S)(+)tamsulosin hydrochloride of less than 0.1% when analyzed by chiral HPLC.

The invention further includes formulations containing (R)(−)tamsulosin hydrochloride, obtained from (R)-tamsulosin free base prepared according to a process of the invention, having a content of (S)(+)tamsulosin hydrochloride of less than 1%.

The invention further includes formulations containing (R)(−)tamsulosin hydrochloride, obtained from (R)-tamsulosin free base prepared according to a process of the invention, having a content of (S)(+)tamsulosin hydrochloride of less than 0.5%.

The invention further includes formulations containing (R)(−)tamsulosin hydrochloride, obtained from (R)-tamsulosin free base prepared according to a process of the invention, having a content of (S)(+)tamsulosin hydrochloride of less than 0.1%.

Additional embodiments of the invention include utilizing tamsulosin base made according to the described process to further produce pharmaceutically acceptable or desirable addition salts, hydrates, solvates, or clathrates of tamsulosin. Preferably, these additional embodiments include forming an acid addition salt of tamsulosin obtained from the above reaction by treating a solution or suspension of that tamsulosin with an appropriate acid. Such processes isolate the tamsulosin product by (i) extracting the tamsulosin product in an organic solvent and (ii) at least partially evaporating the organic solvent to obtain the tamsulosin product, and then reacting the isolated tamsulosin product to produce the tamsulosin salt, hydrate, solvate or clathrate.

Optionally, additional purification steps can be included without altering the invention, such as heating in an organic solvent, filtering, drying, and re-crystallizing in a hot alkanol, such as ethanol.

The invention further includes formulating tamsulosin, its salts and/or in vivo cleavable prodrugs thereof (collectively “the compounds of the invention”) prepared from tamsulosin base made according to the described process, into readily usable dosage units for the therapeutic treatment (including prophylactic treatment) of mammals, including humans. Such formulations are normally formulated in accordance with standard pharmaceutical practice as a pharmaceutical composition. According to this aspect of the invention there is provided a pharmaceutical composition that includes the compounds of the invention, as defined hereinbefore, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as the sodium salt of benzoic acid, ethyl or propyl p-hydroxybenzoate), anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used, and the aerosol device is conveniently arranged to dispense a metered quantity of active ingredient.

The amount of a compound of this invention that is combined with one or more excipients to produce a single dosage form will necessarily vary depending upon the host treated and the particular route of administration. For example, a formulation intended for oral administration to humans may contain, for example, from 0.5 mg to 2 g of active ingredient compounded with an appropriate and convenient amount of excipients which may vary from about 5 to about 98 percent by weight of the total composition. Dosage unit forms will generally contain about 1 mg to about 500 mg of an active ingredient.

The size of the dose for therapeutic or prophylactic purposes of the compounds of the invention will naturally vary according to the nature and severity of the conditions, the age and sex of the animal or patient, and the route of administration, according to well known principles of medicine. For example, the method may comprise at least one of an hourly administration, a daily administration, a weekly administration, or a monthly administration of one or more compositions described herein.

In addition to the compounds of the invention, the invention also includes solvates, pharmaceutically acceptable prodrugs, pharmaceutically active metabolites, and pharmaceutically acceptable salts of such compounds.

The term “solvate” refers to an aggregate of a molecule with one or more solvent molecules.

A “pharmaceutically acceptable prodrug” is a compound that may be converted under physiological conditions or by solvolysis to the specified compound or to a pharmaceutically acceptable salt of such compound.

A “pharmaceutically active metabolite” is a pharmacologically active product produced through metabolism in the body of a specified compound or salt thereof. Metabolites of a compound may be identified using routine techniques known in the art, and their activities determined using tests such as those described herein.

Prodrugs and active metabolites of a compound may be identified using routine techniques known in the art. Various forms of prodrugs are known in the art.

According to the invention, suitable methods of administering the therapeutic composition of the invention to a patient include any route of in vivo administration that is suitable for delivering the composition into a patient. The preferred routes of administration will be apparent to those of skill in the art, depending on the type of condition to be prevented or treated, and/or the target cell population. Preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue.

It will be apparent to those skilled in the art that various modifications and variations can be made in the present invention and specific examples provided herein without departing from the spirit or scope of the invention. Thus, it is intended that the invention covers the modifications and variations of this invention that come within the scope of any claims and their equivalents.

The following examples are for illustrative purposes only and are not intended, nor should they be interpreted to, limit the scope of the invention.

### EXAMPLE 1

### Preparation of 5-[(2R)-2-[[2-(2-ethoxyphenoxy)ethyl]amino]propyl]-2-methoxybenzene-sulfonamide (Tamsulosin Base)

(R)-5-(2-aminopropyl)-2-methoxybenzenesulfonamide (60 g; 245.59 mmol) and 1-(2-bromoethoxy)-2-ethoxybenzene (57.25 g; 233 mmol) were dissolved in 240 mL of N,N-dimethylformamide and 50.5 mL of diisopropylethylamine. The reaction mixture was heated to 100° C. and stirred for 90 minutes. The mixture was then cooled to 20° C. and 720 mL of ethyl acetate and 300 mL of water added to the vessel. The mixture was then stirred for 20 minutes at atmospheric condition, and then was allowed to settle. The aqueous layer was separated and re-extracted twice with 300 mL of ethyl acetate. The organic layers were combined and washed with 600 mL of water.

The organic phase (1120 mL of ethyl acetate) was then concentrated by distillation at atmospheric pressure during which a white solid was precipitated. The mixture was then cooled to 2° C. and stirred for 1 hour. The resulting crystals were isolated by filtration and washed with ethyl acetate to yield 133.59 g of wet tamsulosin base (Loss on Drying: 55.08% (corresponding to 60.01 g of dry material); HPLC Purity: 95.025%).

### EXAMPLE 2

### Preparation of 5-[(2R)-2-[[2-(2-ethoxyphenoxy)ethyl]amino]propyl]-2-methoxybenzene-sulfonamide hydrochloride (Tamsulosin Hydrochloride)

In a round-bottomed flask, 123.66 g (55.54 g dry equivalent weight, 135.95 mmol) of wet 5-[(2R)-2-[[2-(2-ethoxyphenoxy)ethyl]amino]propyl]-2-methoxybenzene-sulfonamide (Tamasulosin base) and 548.06 g of methanol were combined and the mixture was stirred at 50° C. to obtain a complete dissolution. The solution was then cooled to 36±2° C. and any remaining mechanical particles were removed by filtration to yield a clear solution. To the solution was added 14.16 g (135.95 mmol) of hydrochloric acid followed by 459.36 g of methanol. The solution was then distilled at atmospheric pressure and cooled to 2±2° C. during which a white solid precipitated. The resulting crystals were isolated by filtration and washed with methanol. The white product was then suspended in 206.00 g of methanol at 60° C. for 30 minutes and, after being cooled to 2±2° C., the organic solid was isolated by filtration. The solid was then re-suspended, cooled and filtered to yield tamsulosin hydrochloride which was then dried to a constant weight under vacuum at 60° C. (Yield: 46.28 g (104.00 mmols, 76.5 % molar yield). Analysis: Potentiometric Assay using HClO₄: 99.56% %; Melting Point: 227.6-230.1° C.; Chemical Purity (HPLC): 99.81%; Optical Purity (Chiral HPLC): content of (S)(+)tamsulosin=0.125%).

### EXAMPLE 3

### Preparation of 5-[(2R)-2-[[2-(2-ethoxyphenoxy)ethyl]amino]propyl]-2-methoxybenzene-sulfonamide (Tamsulosin Base)

(R)-5-(2-aminopropyl)-2-methoxybenzenesulfonamide (100 g; 409.31 mmol) and 1-(2-bromoethoxy)-2-ethoxybenzene (95.4 g; 388 mmol) were dissolved in 400 mL of N,N-dimethylformamide and 84 mL of diisopropylethylamine. The reaction mixture was heated to 100° C. and stirred for 90 minutes. The mixture was then cooled to 20° C., and 1200 mL of ethyl acetate and 500 mL of water were added. The mixture was then stirred for 20 minutes at atmospheric conditions and was allowed to settle. The aqueous layer was then separated and re-extracted twice with 500 mL of ethyl acetate. Next, the organic layers were combined and washed with 1000 mL of water.

The organic phase (˜1570 mL of ethyl acetate) was then concentrated by distillation at atmospheric pressure during which a white solid was precipitated. The mixture was then cooled to 2° C., and stirred for 1 hour. The resulting crystals were isolated by filtration and washed with ethyl acetate to yield 125.93 g of wet tamsulosin base (Loss on Drying: 24.09% (corresponding to 95.59 g of dry material); HPLC Purity: 97.62%).

### EXAMPLE 4

### Preparation of 5-[(2R)-2-[[2-(2-ethoxyphenoxy)ethyl]amino]propyl]-2-methoxybenzene-sulfonamide (Tamsulosin Base)

(R)-5-(2-aminopropyl)-2-methoxybenzenesulfonamide (3 g; 12.27 mmol) and 1-(2-bromoethoxy)-2-ethoxybenzene (2.86 g; 11.63 mmol) were dissolved in 12 mL of N,N-dimethylformamide and 2.55 mL of diisopropylethylamine. The reaction mixture was heated to 100° C. and stirred for 90 minutes. The mixture was then cooled to 20° C., and 36 mL of ethyl acetate and 15 mL of water were added. The mixture was then stirred for 20 minutes at atmospheric conditions and was allowed to settle. The aqueous layer was then separated and re-extracted twice with 15 mL of ethyl acetate. Next, the organic layers were combined and washed with 75 mL of water.

The organic phase (˜80 mL of ethyl acetate) was concentrated by distillation at atmospheric pressure during which a white solid was precipitated. The mixture was then cooled to 2° C. and stirred for 1 hour. The resulting crystals were isolated by filtration and washed with ethyl acetate to yield 5.14 g of wet tamsulosin base (Loss on Drying: 39.68% (corresponding to 3.1 g of dry material); HPLC Purity: 96.65%).

The solid obtained in the previous step was then combined with 31 mL of ethanol. The reaction mixture was then heated to 78° C. and stirred for 40 minutes, cooled to 0° C. and stirred for 150 minutes. The resulting crystals were isolated by filtration to yield 4.92 g of wet tamsulosin base (Loss on Drying: 40.59% (corresponding to 2.81 g of dry material); HPLC Purity: 98.70%).

### EXAMPLE 5

### Preparation of 5-[(2R)-2-[[2-(2-ethoxyphenoxy)ethyl]amino]propyl]-2-methoxybenzene-sulfonamide hydrochloride (Tamsulosin Hydrochloride)

In a round-bottomed flask, 119.04 g (90.36 g dry equivalent weight, 221.19 mmol) of wet 5-[(2R)-2-[[2-(2-ehtoxyphenoxy)ethyl]amino]propyl]-2-methoxybenzene-sulfonamide (Tamasulosin base) and 1125.79 mL of methanol were combined and stirred at 50° C. to obtain a complete dissolution. The solution was then cooled to 36±2° C., and any remaining mechanical particles were removed by filtration to yield a clear solution. To the solution was added 22.4 g (135.95 mmol) of hydrochloric acid followed by 1034 mL of methanol. The solution was then distilled at atmospheric pressure and cooled to 2±2° C. during which a white solid precipitated. The resulting crystals were isolated by filtration and washed with methanol. The white product was then suspended in 509.1 mL of methanol at 60° C. for 30 minutes and, after being cooled to 2±2° C., the organic solid was isolated by filtration. The solid was then re-suspended, cooled and filtered to yield tamsulosin hydrochloride which was then dried to a constant weight under vacuum at 60° C. (Yield: 46.28 g (199.00 mmols, 84.32% molar yield)). Analysis: Potentiometric Assay using HClO₄: 99.78%; Melting Point:

229.0-230.3° C.; Chemical Purity (HPLC): 99.82%; Optical Purity (Chiral HPLC): content of (S)(+)Tamsulosin=0.08%).

### General Experimental Conditions:

In the various examples described above, the HPLC techniques used were as follows:

### HPLC Method for Chemical Purity:

HPLC tests for measuring chemical purity were performed on a Kromasil C8 4.6×250 mm, 5 μm I.D column, at room temperature (˜20-25° C.). The mobile phase A was prepared by mixing 300 mL of acetonitrile with 700 mL of buffer (pH=3.5) that was prepared from 0.77 g of KH₂PO₄ and 1.2 g of pentanesulfonic acid sodium salt dissolved in 700 mL of water and by adjusting the pH to 3.5 with 10% orthophosphoric acid. The mobile phase B was acetonitrile. The mobile phases were prepared and filtered through a 0.22 μm nylon filter under vacuum.

The chromatograph was programmed as follows: Initial 0-10 minutes isocratic 100% mobile phase A; 10-20 minutes linear gradient to 80% mobile phase A; 20-50 minutes, isocratic 80% mobile phase A; 50-60 minutes, linear gradient to 100% mobile phase A; and 60-70 minutes equilibration with 100% mobile phase A.

The chromatograph was equipped with a 215 nm detector and the flow rate was 1.2 mL per minute. Test samples (10 μl) were prepared by dissolving the appropriate amount of sample in order to obtain 2 mg per mL in mobile phase A.

### HPLC Method for Optical Purity:

HPLC tests for measuring optical purity were performed on a Chiralpack AD-H, 5 μm I.D, 4.6 mm×25 cm column, at room temperature (˜20-25° C.). The mobile phase was prepared by mixing 800 mL of n-hexane and 200 mL of 0.1% diethylamine in 2-propanol solution. This mobile phase was mixed and filtered through a 0.22 μm nylon filter under vacuum.

The chromatograph was equipped with a 225 nm detector and the flow rate was 1.0 mL per minute. Test samples (50 μl) were prepared by dissolving the appropriate amount of sample in order to obtain 0.4 mg per mL of a mixture of 2 volumes of 0.1% diethylamine in 2-propanol solution with 3 volumes of n-hexane.

Although the invention has been described and illustrated with a certain degree of particularity, it is understood that the present disclosure has been made only by way of example, and that numerous changes in the conditions and order of steps can be resorted to by those skilled in the art without departing from the spirit and scope of the invention.

What is claimed is:

1. A process for preparing (R)(−)tamsulosin free base comprising reacting (R)-5-(2-aminopropyl)-2-methoxybenzenesulfonamide or an addition salt thereof with 1-(2-bromoethoxy)-2-ethoxybenzene in at least one polar aprotic solvent and in the presence of at least one organic base.

2. The process of claim 1, further comprising extracting said (R)(−)tamsulosin free base with at least one organic solvent.

3. The process of claim 1, further comprising precipitating said (R)(−)tamsulosin free base.

4. The process of claim 1, further comprising converting said (R)(−)tamsulosin free base into its corresponding hydrochloride salt.

5. The process of claim 2, further comprising at least partially evaporating said at least one organic solvent to obtain (R)(−)tamsulosin free base.

6. The process of claim 1, wherein said (R)(−)tamsulosin free base has a purity higher than approximately 97% when analyzed by reverse phase HPLC.

7. The process of claim 1, wherein said (R)(−)tamsulosin free base is solid.

8. The process of claim 1, wherein said (R)(−)tamsulosin free base is crystalline.

9. The process of claim 1, wherein said at least one polar aprotic solvent is N,N-dimethylformamide.

10. The process of claim 1, wherein said at least one organic base is N,N-diisopropylamine.

11. The process of claim 2, wherein said at least one organic solvent is ethyl acetate.

12. The process of claim 1, wherein the molar ratio of said (R)-5-(2-aminopropyl)-2-methoxybenzenesulfonamide to said 1-(2-bromoethoxy)-2-ethoxybenzene compound is between approximately 0.90 and approximately 1.10.

13. The process of claim 1, wherein the molar ratio of said (R)-5-(2-aminopropyl)-2-methoxybenzenesulfonamide to said 1-(2-bromoethoxy)-2-ethoxybenzene compound is approximately 1.05.

14. Crystalline (R)(−)tamsulosin free base prepared by the process of claim 1, wherein said (R)(−)tamsulosin free base has a purity higher than approximately 97% when analyzed by reverse phase HPLC.

15. Crystalline (R)(−)tamsulosin free base prepared by the process of claim 1, wherein said (R)(−)tamsulosin free base has an X-ray diffraction pattern substantially similar to that of FIG. 1.

16. Crystalline (R)(−)tamsulosin free base prepared by the process of claim 1, wherein said (R)(−)tamsulosin free base has an X-ray diffraction pattern (2θ) having characteristics peaks at approximately 8.56, approximately 13.61, approximately 15.38, approximately 17.25, approximately 18.68 and approximately 22.85 degrees.

17. A process for preparing (R)(−)tamsulosin hydrochloride comprising converting crystalline (R)(−)tamsulosin free base prepared according to the process of claim 1 into (R)(−)tamsulosin hydrochloride.

18. (R)(−)tamsulosin hydrochloride prepared according to the process of claim 17, wherein said (R)(−)tamsulosin hydrochloride has an (S)(+)tamsulosin hydrochloride content of less than approximately 1% when analyzed by chiral HPLC.

19. (R)(−)tamsulosin hydrochloride prepared according to the process of claim 17, wherein said (R)(−)tamsulosin hydrochloride has an (S)(+)tamsulosin hydrochloride content of less than approximately 0.5% when analyzed by chiral HPLC.

20. (R)(−)tamsulosin hydrochloride prepared according to the process of claim 17, wherein said (R)(−)tamsulosin hydrochloride has an (S)(+)tamsulosin hydrochloride content of less than approximately 0.15% when analyzed by chiral HPLC.

21. (R)(−)tamsulosin hydrochloride prepared according to the process of claim 17, wherein said (R)(−)tamsulosin hydrochloride has an (S)(+)tamsulosin hydrochloride content of less than approximately 0.1% when analyzed by chiral HPLC.

22. Formulations containing (R)(−)tamsulosin hydrochloride prepared according to the process of claim 17, wherein said (R)(−)tamsulosin hydrochloride has an (S)(+)tamsulosin hydrochloride content of less than approximately 1%.

23. Formulations containing (R)(−)tamsulosin hydrochloride prepared according to the process of claim 17, wherein said (R)(−)tamsulosin hydrochloride has an (S)(+)tamsulosin hydrochloride content of less than approximately 0.5%.

24. Formulations containing (R)(−)tamsulosin hydrochloride prepared according to the process of claim 17, wherein said (R)(−)tamsulosin hydrochloride has an (S)(+)tamsulosin hydrochloride content of less than approximately 0.15%.

25. Formulations containing (R)(−)tamsulosin hydrochloride prepared according to the process of claim 17, wherein said (R)(−)tamsulosin hydrochloride has an (S)(+)tamsulosin hydrochloride content of less than approximately 0.1%.

26. (R)(−)tamsulosin hydrochloride having a purity higher than approximately 99% when analyzed by reverse phase HPLC.

27. (R)(−)tamsulosin hydrochloride having a purity higher than approximately 99.5% when analyzed by reverse phase HPLC.

28. (R)(−)tamsulosin hydrochloride having a purity higher than approximately 99.9% when analyzed by reverse phase HPLC.
